(19) United States
(12) Patent Application Publication
(10) Pub. No.: US 2018/0271826 A1
(43) Pub. Date: Sep. 27, 2018

(54) DRY POWDERS OF CANNABINOIDS AND METHODS FOR PREPARING DRY POWDERS

(71) Applicants: Robert E. Sievers, Boulder, CO (US); Lia Rebits, Thornton, CO (US)

(72) Inventors: Robert E. Sievers, Boulder, CO (US); Lia Rebits, Thornton, CO (US)

(73) Assignee: COLORADO CAN LLC, Boulder, CO (US)

(21) Appl. No.: 15/466,719

(22) Filed: Mar. 22, 2017

(51) Int. Cl. A61K 31/352; A61K 9/14; A61K 9/00; A61K 31/05; A61K 9/70

(52) U.S. Cl. CPC A61K 31/352; A61K 9/146; A61K 9/7007; A61K 9/145; A61K 31/05; A61K 9/0078

(57) ABSTRACT

Dry powders comprise a cannabinoid, a polymer binding agent, a dispersing agent, and a bulking agent, and optionally an antioxidant, and are formed by carbon dioxide-assisted nebulization and drying in a flowing dry stream of gas. The dry powders have an aerodynamic particle distribution effective for delivery by respiration into the lungs of a patient. Methods of preparing a dry powder comprise subjecting a solution of a volatile component, a cannabinoid, a polymer binding agent, a dispersing agent, and a bulking agent, and optionally an antioxidant, to carbon dioxide-assisted nebulization, and drying droplets formed by the nebulization in a flowing dry gas stream to form a dry powder. The dry powder has an aerodynamic particle distribution effective for delivery of the dry powder by respiration into a lung of a patient and/or exhibit increased bioavailability and/or storage stability of a cannabinoid. The dry powder can be compressed into a wafer.

### FIELD OF THE INVENTION

The present invention is directed to dry powders comprising one or more cannabinoids and to methods of preparing such dry powders. In additional embodiments, the invention is directed to dry powders comprising one or more cannabinoids which provide improved stability and/or bioavailability as compared with previous cannabinoid compositions, and to methods of preparing such dry powders.

### BACKGROUND OF THE INVENTION

Cannabinoids in the form of cannabis and cannabis extracts have been used for centuries for both medicinal and recreational purposes, but only relatively recently have the pharmacokinetics and potential clinical uses of these compounds been explored scientifically. Knowledge of the psychoactive effects of cannabis appears to have originated in the Himalayan region and spread to India, Africa, and eventually to Europe (Kalant 2001). In the 19^{th} and early 20^{th} centuries, cannabis extracts were widely used in the English-speaking world as sedative, hypnotic and anticonvulsant agents, and in fact were described in both the British and American Pharmacopoeias (Walton 1938, Mikuriya 1969). By the mid-20^{th} century, however, cannabis preparations had been removed from both Pharmacopoeias, due largely to the fact that “the plant material was too variable in composition, its shelf-life was too short and unpredictable, and it had been increasingly replaced by pure opiates and more reliable new synthetic drugs invented in the early part of the 20^{th} century” (Kalant 2001).

Despite the variability of cannabis preparations that led to their official removal from the Pharmacopoeias, the active ingredients in cannabis, the cannabinoids, show great promise as therapeutic drugs. Prominent among the claimed clinical utilities of cannabis is the use as an analgesic, and several short term trials have demonstrated reductions in postoperative (Jain 1981), dental (Raft 1977), cancer (Noyes 1975), and visceral (Holdcroft 1997) pain. The central nervous system action of Δ9-tetrahydrocannabinol (THC), which binds to CB1 receptors, is separate from the action of opioid analgesics, and produces reduction of pain and an increase in pain tolerance (Milstein 1975, Meng 1998, Fuentes 1999). In addition to pain management, cannabis preparations have well-documented antinauseant and antiemetic properties (Brown 1998, Hartel 1999), as well as potent appetite-stimulating effects (Jones 1976, Mendelson 1976, Mattes 1994).

Both THC and cannabidiol (CBD) have electrophysiological effects similar to the anti-seizure medicine phenytoin (Chiu 1979, Karler 1981, Consroe 1982), and the well-known decrease in skeletal muscle tone and ataxia that is centrally mediated by cannabis appears to be accompanied by a peripherally controlled antispasticity action (Consroe 1998). Another musculature effect caused by cannabis is smooth muscle relaxation, which leads to bronchodilation upon inhalation of a cannabis preparation. Often, however, the acute bronchodilation effect is offset by chronic irritation of the airways by particulates when the cannabis is smoked (Tashkin 1999). Lastly, when cannabis is administered systemically, or less consistently, locally, into the eye, a decrease in the intraocular pressure has been repeatedly observed (Green 1998).

Research on cannabinoid pharmacokinetics reveals that the most common routes of administration of marijuana or marijuana-based preparations (smoking, vaping, ingestion, transcutaneous diffusion) are inefficient at delivering cannabinoids contained in the preparation due to low bioavailability, cannabinoid degradation, or both. Smoking, though a rapid method of drug delivery, provides only 2 to 56% bioavailability of the total cannabinoid content of the smoked material due to variability in smoking dynamics, such as number and frequency of puffs, inspiration volume, hold time, etc. (Widman 1971, Ohlsson 1985, Agurell 1986, Ohlsson 1982). Another contributing factor to low bioavailability of smoked cannabinoids appears to be degradation during the combustion process. Experimentally, when the total smoke and remaining ash were collected during the burning of cannabis cigarettes, only 36.9% and 38.4% of the theoretically present THC and cannabidiol (CBD), respectively, were recovered; the missing mass balance was attributed to degradation of the cannabinoids during combustion (Elzinga 2015).

An alternative cannabinoid active ingredient is synthetic THC, known by its generic name, dronabinol, and commercially available as Marinol® from AbbVie in the form of soft gelatin capsules containing either 2.5 mg, 5 mg, or 10 mg dronabinol. The capsules are formulated with gelatin, glycerin, and sesame oil for gastrointestinal oral delivery in treatment of nausea. However, owing to poor storage stability of the formulations, it is recommended that the capsules should be stored in a cool environment between 8° and 15° C. (46° and 59° F.) or alternatively should be stored in a refrigerator. Additionally, the Marinol® prescribing information indicates that due to the combined effects of first pass hepatic metabolism and high lipid solubility, only 10 to 20% of an administered dose reaches the systemic circulation.

Dronabinol has also recently been approved by US FDA in liquid form under the name Syndros from Insys Therapeutics, Inc. for nausea and vomiting associated with cancer chemotherapy in patients who have failed to respond adequately to conventional antiemetic treatments and for anorexia associated with weight loss in patients with AIDS. Insys indicates that Syndros should be refrigerated at 2-8° C. (36-46° F.), with excursions permitted to 15-25° C. (59-77° F.). An opened bottle can be stored at 25° C. (77° F.) for 28 days after first opening; however, any unused portions thereafter should be discarded. Syndros is indicated for oral administration, with the same first pass hepatic metabolism of the active dronabinol as with Marinol® significantly limiting the amount of an administered dose which reaches the systemic circulation.

More specifically, delivery of cannabinoids via the oral route by ingestion is reported to confer a similarly low bioavailability of 10-20% (Kim 1996), with peak Δ9-tetrahydrocannabinol (THC) concentrations occurring 1-5 hours after dosing (Ohlsson 1980). Much of the low oral bioavailability can be attributed to incomplete and variable absorption in the gastrointestinal tract that depends on stomach contents and cannabinoid vehicle (Perez-Reyes 1973), coupled with a further reduction in the successfully absorbed quantity due to oxidation by the liver before the cannabinoid can reach the brain (Mattes 1993). Transdermal delivery of cannabinoids, administered by a patch placed on the skin, has been shown to be a very mild dosage form, with peak THC concentrations of only 4.4 ng/ml after 1.4 hours (Stinchcomb 2004), and is incompatible with rapid delivery of larger doses.

Degradation during storage and variable bioavailability lead to considerable dosage inconsistency from the standpoint of the patient, and are major confounding factors in the design of clinical trials. A meta-analysis of the treatment of chemotherapy side effects such as nausea and emesis in 750 patients revealed that 65% of the cases presented with a poor response to treatment from orally administered cannabis-based therapies (Plasse 1991). The low and/or variable bioavailability of ingested cannabinoids may be a significant causative factor to poor response to treatment (Huestis 2007). Reliable, consistent, and rapid delivery of high doses of cannabinoids has so far only been demonstrated in intravenous (Agurell 1986) or rectal (El Sohly 1991) administration of cannabis-based therapeutics. Such delivery routes are not feasible or acceptable for the majority of patients.

In concert with the low bioavailability provided by common routes of administration, degradation of cannabinoids during storage also commonly contributes to cannabinoid waste, production inefficiency, and inconsistent dosage. When stored in the preparations most commonly encountered (resin, oil, dried plant material, tincture), significant cannabinoid degradation can occur, even when the preparation is stored at cool temperatures and protected from light. A study of the degradation of cannabis resin demonstrated that after 4 years, 95.77% of the original THC content was degraded, even during storage at 4° C. in the dark, and the CBD content in the same resin was reduced by 55.75% (Trofin 2012). Under less forgiving storage conditions, such as room temperature (20° C.) near a source of light, THC degradation is more rapid, with one study reporting 37% of the original THC content in plant material remaining after a little under two years (Fairbarn 1976).

In view of the storage instability of cannabinoids, including the commercially available dronabinol, and the relatively low bioavailability of orally administered formulations, there is a great need for alternative formulations for cannabinoid delivery that provide improved patient convenience of administration, improved storage stability and/or improved bioavailability. There is also a great need for cannabinoid therapeutic products which are safer, more accurate and more reliable than those administered by combustion, and for such therapeutic products that can provide rapid delivery of cannabinoids to the bloodstream for expeditious relief.

### SUMMARY OF INVENTION

It is therefore an object of the invention to provide new formulations for administration of cannabinoids. It is a further object of the invention, in certain embodiments, to provide new formulations for administration of cannabinoids which overcome one or more problems of known formulations and methods for administration of cannabinoids.

In one embodiment, the invention is directed to a dry powder which comprises a cannabinoid, a polymer binding agent, a dispersing agent, and a bulking agent, and, optionally, an antioxidant. The dry powder is formed by carbon dioxide-assisted nebulization and drying in a flowing stream of dry gas. The dry powder has an aerodynamic particle distribution effective for delivery of the dry powder by respiration into the lungs of a patient.

In another embodiment, the invention is directed to a method of preparing a dry powder comprising a cannabinoid. The method comprises subjecting a solution of a volatile component, a cannabinoid, a polymer binding agent, a dispersing agent, and a bulking agent, and, optionally, an antioxidant, to carbon dioxide-assisted nebulization, and drying droplets formed by the nebulization in a flowing gas stream to form a dry powder. The dry powder has an aerodynamic particle distribution effective for delivery of the dry powder by respiration into the lungs of a patient.

In yet another embodiment, the invention is directed to a method of increasing the bioavailability and/or storage stability of a cannabinoid. The method comprises subjecting a solution of a volatile component, a cannabinoid, a polymer binding agent, a dispersing agent, and a bulking agent, and, optionally, an antioxidant, to carbon dioxide-assisted nebulization, and drying droplets formed by the nebulization in a flowing dry gas stream to form a dry powder.

The dry powders and methods according to the invention are advantageous in providing a formulation for convenient and reliable pulmonary administration of a cannabinoid to a patient. In certain embodiments, the dry powders according to the invention are advantageous in providing a formulation which exhibits improved stability and/or bioavailability of the cannabinoid active ingredient. These and additional advantages of the invention will be more evident in view of the following detailed description of the invention.

### BRIEF DESCRIPTION OF DRAWINGS

The detailed description of the invention will be more fully understood in view of the drawings, in which:

FIG. 1 shows a schematic diagram of a carbon dioxide-assisted nebulization with bubble drying (CAN-BD) process which may be used in the methods of the invention (Sellers 2001).

FIG. 2 shows the Andersen Cascade Impactor apparatus and a correlation of the stages with the anatomical areas of the human respiratory system (Andrade-Lima 2012). Tests done using this method are in compliance with the United States Pharmacopoeia (USP), Monograph <601> Inhalation and Nasal Drug Products: Aerosols, Sprays, and Powders, Performance Quality Tests.

FIG. 3 shows the delivery of THC to the pharynx and lung (fine particle formation (FPF)<5.8 μm) as modeled and fractionated by an Andersen Cascade Impactor (USP <601>) and assayed by high performance liquid chromatography (HPLC), with a commercial product for comparison, and three dry powder inhalers employing dry powders according to the invention.

The drawings show certain features related to the invention but are not to be construed as limiting of the invention in any manner.

### DETAILED DESCRIPTION

The present invention provides new formulations for administration of cannabinoids, and, in certain embodiments, provides new formulations for administration of cannabinoids which overcome one or more problems of known formulations and methods for administration of cannabinoids. Throughout this specification, when a range of values is defined with respect to a particular characteristic of the present invention, the present invention relates to and explicitly incorporates every specific subrange therein. Additionally, throughout this specification, when a group of substances is defined with respect to a particular characteristic of the present invention, the present invention relates to and explicitly incorporates every specific subgroup therein. Any specified range or group is to be understood as a shorthand way of referring to every member of a range or group individually as well as every possible subrange and subgroup encompassed therein.

Despite low bioavailability of the total cannabinoid content of smoked material, delivery of cannabinoids to the lungs is an attractive route of administration. The high lipid-solubility of cannabinoids allows them to cross the alveolar membrane rapidly and enter the pulmonary capillaries, from which they are carried to the heart and quickly pumped to the brain. This expedient delivery results in an onset of benefits at least as rapid as intravenous injection (Kalant 2001). Pharmaceutical delivery methods like pulmonary inhalation, that by-pass the gastrointestinal tract and liver are said to provide one free pass in avoiding oxidation or degradation therein, and therefore provide an improvement in bioavailability over oral administration.

The dry powders and methods therefore may provide solutions to the prior art problems of low bioavailability of cannabinoids by presenting the cannabinoid in a novel dry, inhalable powder. By co-processing the cannabinoid active ingredient with selected excipients, a fine dry microparticulate powder with the correct aerodynamic particle size distribution and adequate dispersibility for inhalation into the lung and pharynx is achieved. Delivery of cannabinoids to the lung via a dry powder provides a similarly rapid increase in peak plasma cannabinoid levels as achieved by smoking, without simultaneous delivery of carcinogens or other unacceptable combustion products. Unlike smoking, the dose of active ingredient in the aerosolized dry powder can be precisely controlled, and, as a result, dose variability is eliminated by administration of the powder with an appropriate dry powder inhaler (DPI). Such respiratory cannabinoid delivery avoids the poor gastrointestinal tract absorption and first-pass metabolism issues that occur during oral administration, and is suitable to deliver larger quantities of active ingredient more rapidly than transdermal and most other methods of dosing.

In addition to enabling delivery to the respiratory system, the excipients which are co-processed with the cannabinoid can stabilize the preparation during storage and mitigate oxidative or other chemical processes that cause degradation of the active ingredients. Stabilizing excipients and/or opaque containers can also serve to shield the cannabinoids from light, thus reducing the amount of degradation due to reactions which occur in ultraviolet light.

The dry powders according to the invention are formed by subjecting a solution of a volatile component, a cannabinoid, a polymer binding agent, a dispersing agent, and a bulking agent, and, optionally, an antioxidant, to carbon dioxide-assisted nebulization, and drying droplets formed by the nebulization in a flowing dry gas stream to form a dry powder. Generally, in one embodiment, the methods employ supercritical or near critical carbon dioxide, or a mixture of supercritical or near critical carbon dioxide with one or more other supercritical or near critical substances, and comprise (a) forming a composition comprising a volatile component, a cannabinoid, a polymer binding agent, a dispersing agent, and a bulking agent, and, optionally, an antioxidant, and the supercritical or near critical carbon dioxide; (b) reducing the pressure on the composition, whereby droplets are formed; and (c) passing the droplets through a flow of drying gas which is not the supercritical or near critical carbon dioxide. A suitable drying gas is, for example, gaseous nitrogen or gaseous carbon dioxide (i.e., not under supercritical or near critical conditions). The drying gas is heated from above ambient temperature to about 40° C. Carbon dioxide-assisted nebulization with a bubble dryer (CAN-BD) is a proprietary process described by Sellers 2001 and Sievers et al, U.S. Pat. No. 6,630,121 B1, each of which is incorporated herein by reference in its entirety, and is suitable for use in the present methods. CAN-BD produces extremely fine, stable dry powders from the compositions disclosed herein in a desirable particle size range for inhalation. The drying step is conducted at lower temperatures than traditional spray drying which minimizes the thermal decomposition of labile materials, and is complete in seconds, in contrast with the hours often required for lyophilization. The powders produced have a very low moisture content that is compatible with long-term storage stability, and in many cases the CAN-BD process renders the sample amorphous due to the very rapid drying time, potentially increasing the bioavailability compared to crystalline products.

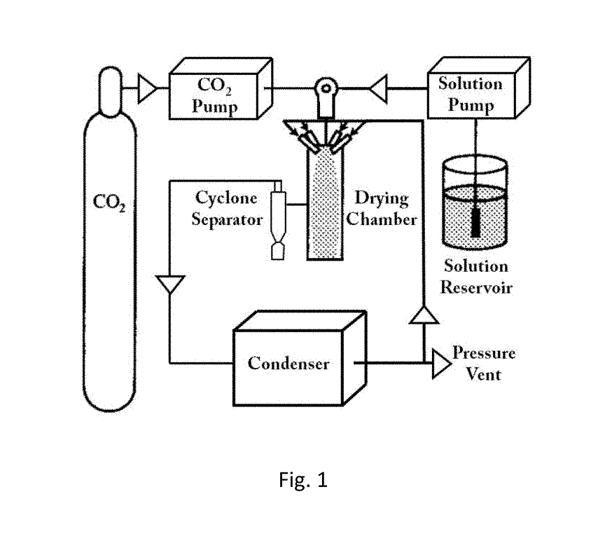

The CAN-BD process is shown schematically in FIG. 1 and employs a unique nozzle configuration as described in U.S. Pat. No. 6,630,121, comprising the combination of a low dead-volume tee and a micro-bore pressure restrictor. Carbon dioxide is compressed into a near-critical liquid or a supercritical fluid with a high-pressure pump and mixed with the solution or suspension containing the components to be dried within the low dead-volume tee to form a micro-emulsion. The emulsion is composed of micron or nanometer diameter droplets and bubbles of solution suspended in fluid carbon dioxide, and the emulsion flows at high pressure down the micro-bore restrictor before rapidly expanding to atmospheric pressure, causing the carbon dioxide to vaporize and the solution droplets to be exposed to a flow of warm drying gas, suitably nitrogen. The solvent is quickly dried from the droplet or bubble, leaving a small particle of solute which falls onto a filter or into a cyclone-separator collection jar and is collected as a dry powder.

Various cannabinoids may be employed in the dry powders of the invention, alone or in a combination of two or more cannabinoids. THC, as the synthetic dronabinol or otherwise produced, cannabidiol, or other cannabinoids may be used, singly or in combination. In a specific embodiment, each cannabinoid that is employed is provided in a pure form, containing less than 1 wt %, more specifically, less than 0.5 wt %, and more specifically, less than 0.1 wt %, impurities. In one embodiment, cannabidiol is provided in a purified form as described in Sievers et al, US 2016/0228385 A1, incorporated herein by reference in its entirety. In a specific embodiment, the cannabinoid, after CAN-BD processing, is rendered amorphous, as described in Sievers et al, US 2016/0228385 A1. Use of formulated amorphous, rather than crystalline state, powders may increase the rate of CBD dissolution in aqueous environments such as the stomach, sublingual mucosal tissue surfaces, or the lungs, and increase bioavailability, in contrast to a slower-dissolving crystalline forms that are often encountered. In addition, certain excipients such as glassy sugars have been proven to enhance the stability of cannabinoids such as Δ9-tetrahydrocannabinol (THC) that are notorious for their limited shelf life (Drooge 2004). Incorporation of these sugars into cannabinoid-containing powders is simple using the CAN-BD process.

The dry powder may contain any suitable effective amount of cannabinoid for a desired therapeutic effect. In a specific embodiment, the dry powder comprises from about 1 to about 95 wt % of cannabinoid. In a more specific embodiment, the dry powder comprises from about 10 to about 40 wt % of cannabinoid. In yet a more specific embodiment, the dry powder comprises from about 30 to about 40 wt % of cannabinoid. Additionally, the dry powder may contain the cannabinoid in an amount sufficient to provide a desired therapeutic dosage when administered from an inhaler, for example, a metered dose or unit dose inhaler. Suitable dosages include from about 0.1 to about 50 mg, more specifically from about 1 to about 10 mg, and even more specifically from about 1 to about 4 mg.

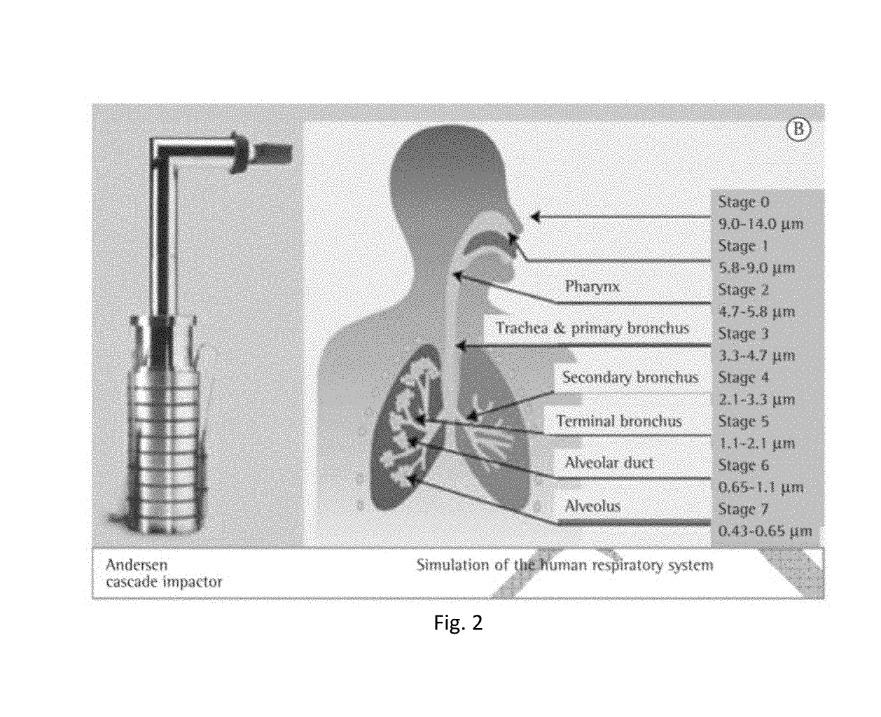

Judicious use of excipients that are co-processed with the cannabinoid can further improve powder properties, storage stability, bioavailability, and/or palatability. Formulation with excipients can greatly affect many properties of an inhalable powder such as its crystallinity and/or storage stability. However, a particularly important property of the present dry powders is the ability of the powders to reach the lung, where the therapeutic dose of inhalable cannabinoid is to be delivered. Many dry powders that appear fine enough to the naked eye to be inhalable are, in fact, composed of such large particles or agglomerations of smaller particles that they are not capable of reaching the lung in significant amounts. If the individual particles do not possess the correct aerodynamic diameter and dispersibility when delivered with a given dry powder inhaler, the powder will impact at the back of the throat and be swallowed into the gastrointestinal tract instead of being inhaled into the lung. Even pressurized metered dose inhalers designed to deliver medication by inhalation often succeed at delivering only 10% of the metered dose to the lung (Wolff 1994). In specific embodiments of the dry powders of the invention, at least 30%, or, more specifically, at least 40%, of particles have a size of less than 5.8 μm as modeled by an Andersen Cascade Impactor according to US Pharmacopeia <601>. Preliminary tests utilizing Andersen Cascade Impaction as described in FIG. 2 with a placebo powder produced by CAN-BD significantly exceed the performance of a typical metered dose inhaler by delivering 40% of the dose to the lung when placed in a metered dose inhaler. This improvement in the fine particle fraction is likely due to the low density of the CAN-BD-produced dry powder as compared to higher density solution droplets or dense crystals emitted by typical metered dose inhalers, which have a greater momentum that is incompatible with sufficient entrainment in the inspiratory air flow for deep lung delivery.

The dry powders of the invention include a polymer binding agent to facilitate the dry powder formation. Without the binding agent, the formulation often forms an “eggshell” or incompletely-dry layer of waxy residue on the collecting filter or cyclone-separator collection jar. Suitable polymer binding agents include polyvinylpyrrolidone (PVP), polyethylene glycol (PEG), poly(lactic-co-glycolic) acid (PLGA). Linear PVP of all of various molecular weights may be employed, while cyclic PVP should not be used unless its safety has been demonstrated. Additional polymer binding agents suitable for use in the dry powders include, but are not limited to, polyvinyl alcohol (PVA), polyacrylic acid (PAA), N-(2-hydroxypropyl) methacrylamide (HPMA), polyoxazoline, polyphosphazenes, xanthan gum, gum arabic, pectins, chitosan derivatives, dextrans, carrageenan, guar gum, cellulose ethers, hyaluronic acid, albumin, and starch. In a specific embodiment, the polymer binding agent comprises polyvinylpyrrolidone having a weight average molecular weight of from about 1000 to about 100,000, or more. The binding agent may also include one or more of lecithin, 1,2-dipalmitoyl-sn-glycero-3-phosphocholine (DPPC), a nonionic surfactant such as Tergitol, and/or a polyphosphate. One or two or more of the indicated binding agents in combination may be used in the inventive dry powders.

The polymer binding agent is employed in the dry powder in an amount sufficient to improve dry powder formation. In a specific embodiment, the dry powder comprises from about 1 to about 30 wt % of polymer binding agent. In a more specific embodiment, the dry powder comprises from about 5 to about 20 wt % of polymer binding agent. In yet a more specific embodiment, the dry powder comprises from about 10 to about 15 wt % of polymer binding agent.

To increase the dispersibility of the powder so that it can be effectively inhaled into the lung, one must often create particles of low density and irregular surface geometry such that the cohesive interactions between particles are reduced. Accordingly, a volatile component is introduced into the solution or suspension used in the dry powder formation, and the resultant dry powder particles contain voids left behind by the sublimation of the salt upon drying. These hollow regions effectively interfere with hydrophobic, hydrophilic, and/or static interactions between particles and allow them to be dispersed more readily than solid particles of the same geometric diameter formed in the absence of the volatile component. In addition, the partially hollow particles possess a lower density than their solid counterparts, allowing them to become better entrained in inspiratory airflow, leading to deeper deposition in the lungs.

Suitable volatile components for use in the present invention include, but are not limited to, volatile salts such as ammonium carbonate, ammonium bicarbonate, triethylammonium bicarbonate, trimethylammonium carbonate, trimethylammonium bicarbonate, ammonium acetate, triethylammonium acetate, trimethylammonium acetate, ammonium formate, trimethylammonium formate, and triethylammonium formate, and volatile oils such as perfluorocarbons, perflubron, and “essential oils” such as alpha-pinene, camphene, sabinene, beta-pinene, beta-myrcene, delta-3-carene, alpha-phellandrene, alpha-terpinene, limonene, eucalyptol, cis-ocimene, gamma-terpinene, terpinolene, fenchone, linalool, camphor, borneol, and geraniol. Any one or two or more volatile components may be used in combination in the inventive dry powders. In a specific embodiment, the volatile component comprises

The volatile component is employed in the dry powder-forming solution in an amount sufficient to provide the dry powder particles with non-uniform shapes and/or porosity or voids to improve dispersibility of the formed dry powder and delivery of the dry powder to the lungs of a patient. In a specific embodiment, the solution comprises from about 0.08 to about 5 wt % of volatile component. In a more specific embodiment, the solution comprises from about 0.08 to about 1 wt % of volatile component. In yet a more specific embodiment, the solution comprises from about 0.16 to about 0.24 wt % of volatile component.

Another additive useful for increasing the dispersibility of the dry powder cannabinoid formulations is a dispersing agent. Suitable dispersing agents comprise amino acids which act as surfactants, including methionine, alanine, arginine, asparagine, aspartic acid, cysteine, glutamic acid, glutamine, glycine, histidine, isoleucine, leucine, lysine, phenylalanine, proline, serine, threonine, tryptophan, tyrosine, and valine. Additional dispersing agent surfactants include dipalmitoylphosphatidycholine (DPPC), phosphatidic acid (PA), phosphatidylethanolamine (PE), phosphatidylcholine (PC), phosphatidylserine (PS), phosphatidylglycerol (PG), Tween 20, and Tween 80. In a specific embodiment, the dispersing agent comprises methionine.

Additionally, the dry powders include a bulking agent, and, specifically, a non-hygroscopic bulking agent. Importantly, the dry powders should not contain any hygroscopic excipients that will retain water on storage or after exposure to ambient conditions. The non-hygroscopic bulking agent aids to prevent significant and unacceptable water retention in the dry powder and to preserve the powder dispersibility. In one embodiment, the bulking agent comprises a non-hygroscopic polyol such as mannitol in an amount sufficient to reduce the overall hygroscopicity of the powder and improve storage stability. Lack of water retention upon storage is advantageous not only for the chemical stability of the cannabinoid active ingredient, but also is essential for the maintenance of the aerodynamic particle size distribution of the powder.

In addition to mannitol, other sugars and sugar alcohols may also be employed as the bulking agent and include one or more of gum Arabic, monosaccharides such as glucose, galactose, fructose, mannose, allose, altrose, fucose, gulose, sorbose, tagatose, arabinose, lyxose, rhamnose, ribose, xylose, erythrose, and threose, disaccharides such as lactose, maltose, sucrose, trehalose, lactulose, cellobiose, chitobiose, allolactose, sucralose, and mannobiose, and polyols such as maltitol, sorbitol, xylitol, erythritol, isomalt, arabitol, ribitol, galactitol, fucitol, iditol, myo-inositol, volemitol, lactitol, maltotriitol, maltotetraitol, maltodextrin, and polyglycitol.

In a specific embodiment, the dry powder comprises from about 10 to about 90 wt % of bulking agent. In a more specific embodiment, the dry powder comprises from about 20 to about 80 wt % of bulking agent. In yet a more specific embodiment, the dry powder comprises from about 30 to about 40 wt % of bulking agent.

An optional component useful for increasing the storage stability of the dry powder cannabinoid formulations is an antioxidant. Suitable antioxidants include molecules that inhibit the oxidation of other molecules. If the dispersing agent as discussed above comprises methionine, which itself exhibits antioxidant properties, the need for an additional antioxidant is reduced. However, in the event that an additional antioxidant is employed, suitable examples include, but are not limited to, include vitamin A, vitamin C, vitamin E, alpha-carotene, astaxanthin, beta-carotene, canthaxanthin, lutein, lycopene, zeaxanthin, flavonoids (such as apigenin, myricetin, eriodictyol, theaflavin, genistein, resveratrol, malvidin), cinnamic acid, chicoric acid, chlorogenic acid, rosmarinic acid, curcumin, xanthones, eugenol, citric acid, oxalic acid, and lipoic acid. In a specific embodiment, the dispersing agent comprises methionine and an additional antioxidant is not employed.

The antioxidant, when employed in the dry powder, is included in an amount sufficient to improve the storage stability of the dry powder. In a specific embodiment, the dry powder comprises from about 1 to about 30 wt % of antioxidant. In a more specific embodiment, the dry powder comprises from about 1 to about 20 wt % of antioxidant. In yet a more specific embodiment, the dry powder comprises from about 5 to about 10 wt % of antioxidant.

The dry powders and methods of the invention encompass any and all combinations of the thus described components within the scope of the general descriptions herein. In specific embodiments, the dry powders and methods employ THC as the cannabinoid, alone or in combination with one or more additional cannabinoids. In further specific embodiments, the dry powders and methods employ cannabidiol as the cannabinoid, alone or in combination with one or more additional cannabinoids. In further embodiments, the polymer binding agent comprises polyvinylpyrrolidone, polyethylene glycol and/or poly(lactic-co-glycolic) acid, the volatile component is a volatile salt, more specifically, ammonium bicarbonate or ammonium carbonate, the dispersing agent is an amino acid, and the bulking agent is a non-hygroscopic polyol. In more specific embodiments, the polymer binding agent comprises polyvinylpyrrolidone, the volatile component is a volatile salt, more specifically, ammonium bicarbonate or ammonium carbonate, the dispersing agent is methionine, and the bulking agent is mannitol.

Finally, in a further embodiment of the invention, a dry powder according to the invention may be compressed into a thin wafer form. Generally, such thin wafers have a thickness of not greater than about 2 mm, or, more specifically, a thickness of about 1 mm. The wafer may be in the shape of a disk, square, ellipsoid, banana, or other configuration, as desired, and may have a diameter or length and width dimensions in the range of about 4-10 mm. In one embodiment, the wafer has a disk configuration with a diameter of about 6-8 mm and a thickness of about 1 mm. In another embodiment, the wafer dissolves in water at room temperature in less than one minute. The wafers may be formed by pressing with conventional equipment at pressures in a range of about 10 to less than about 100 psi, more specifically about 10 to about 80 psi, or about 10 to about 50 psi, or at pressures greater than about 100 psi, or up to about 500, about 1000 or about 2000 psi, i.e., in the range of about 500-2000 psi, or, more specifically, about 1000 psi.

In a further embodiment, the wafer may be provided with a flavorant or fragrance additive in order to increase the palatability of the wafer. In a specific embodiment, the wafer may be provided with a terpene as a flavorant or fragrance additive in order to increase the palatability of the wafer. Suitable terpenes include molecules naturally found in cannabis preparations that are removed by the CAN-BD process, specifically, limonene. Additional terpenes include alpha-pinene, camphene, sabinene, beta-myrcene, delta-3-carene, alpha-phellandrene, alpha-terpinene, eucalyptol, cis-ocimene, trans-ocimene, gamma-terpinene, terpinolene, fenchone, linalool, sabinene hydrate, camphor, isopulegol, isoborneol, borneol, hexahydrothymol, menthol, fenchol, terpineol-4-ol, nerol, alpha-terpineol, geraniol, valencene, pulegone, beta-caryophyllene, alpha-humulene, cis-farnesene, trans-farnesene, guaiol, caryophyllene oxide, nerolidol, cedrol and alpha-bisabolol. In a specific embodiment, the terpene comprises limonene. The terpene may be applied to the wafer by allowing the liquid terpene to diffuse into the pre-compressed wafer, i.e., by dropping it slowly onto the wafer with a pipet. Other methods for applying a liquid terpene to a formed wafer may be employed.

Various aspects of the dry powders and methods of the invention are illustrated in the following Examples.

### Example 1

Dry inhalable powder according to the invention was prepared as described herein and modeled and fractionated by Andersen Cascade Impaction (ACI) and assayed by high performance liquid chromatography (HPLC).

A THC-containing material BC, reported by the manufacturer to have 80% w/w total THC content, was decarboxylated by placing 200 mg into a glass vial and heating in an oven at 110-115° C. for 110 min. The resulting material was resuspended in 1 ml of methanol by sonication for 15 min.

A methanol/water solution (7:3 methanol:water) comprising 3.2% w/w total dissolved solids was made, half of which was ammonium bicarbonate. The remaining dissolved solids were composed of 40% w/w of the described decarboxylated material, 10% methionine, 10% PVP (wt average molecular weight 55 kDa), and 40% mannitol. The solution was dried using the previously described CAN-BD process with the following parameters: 4.5 ml/min. carbon dioxide flow rate, 1.29 ml/min. solution flow rate, 40° C. nitrogen drying gas temperature, 30 L/min. nitrogen drying gas flow rate, 75 μm internal diameter fused silica restrictor, 5 cm long fused silica restrictor, and 0.45 μm nylon powder-collection filter.

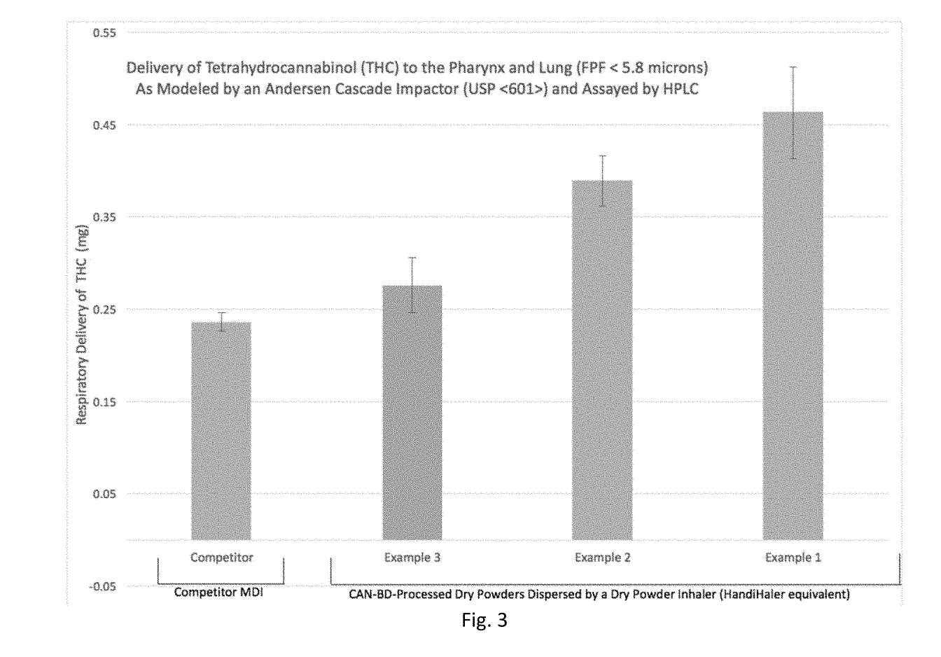

The resulting dry powder fine particle fraction of less than 5.8 μm was 19%, and resulted in a deposition of 0.46+/−0.05 mg of THC into the lung/pharynx region of the respiratory system, as modeled and fractionated by Andersen cascade impaction (ACI) and assayed by high performance liquid chromatography (HPLC) (see FIG. 3.)

### Example 2

Dry inhalable powder according to the invention was prepared as described herein and modeled by Andersen Cascade Impaction (ACI) and assayed by high performance liquid chromatography (HPLC).

THC-containing material “H”, reported by the manufacturer to have 92% w/w total THC content, was dissolved in a minimum amount (about 1 ml) of methanol.

A methanol/water solution (7:3 methanol:water) comprising 3.2% w/w total dissolved solids was made, half of which was ammonium bicarbonate. The remaining dissolved solids were composed of 40% w/w of the described dissolved material, 10% methionine, 15% PVP (wt average molecular weight 55 kDa), and 35% mannitol. The solution was dried using the previously described CAN-BD process with the following parameters: 4.5 ml/min. carbon dioxide flow rate, 1.29 ml/min. solution flow rate, 40° C. nitrogen drying gas temperature, 30 L/min. nitrogen drying gas flow rate, 75 μm internal diameter fused silica restrictor, 5-cm long fused silica restrictor, and 0.45 μm Nylon powder-collection filter.

The resulting dry powder fine particle fraction of less than 5.8 μm was 18%, and resulted in a deposition of 0.39+/−0.03 mg of THC into the lung/pharynx region of the respiratory system, as modeled and fractionated by Andersen cascade impaction (ACI) and assayed by high performance liquid chromatography (HPLC) (see FIG. 3.)

### Example 3

Dry inhalable powder according to the invention was prepared as described herein and modeled by Andersen Cascade Impaction (ACI) and assayed by high performance liquid chromatography (HPLC).

THC-containing material “R”, reported by the manufacturer to have 90% w/w total THC content, was dissolved in a minimum amount (about 1 ml) of methanol.

A methanol/water solution (7:3 methanol:water) comprising 2.1% w/w total dissolved solids was made, 23% of which was ammonium bicarbonate. The remaining dissolved solids were composed of 30% w/w of the described dissolved material, 10% methionine, 10% PVP (wt average molecular weight 55 kDa), and 50% mannitol. The solution was dried using the previously described CAN-BD process with the following parameters: 4.5 ml/min. carbon dioxide flow rate, 1.29 ml/min. solution flow rate, 40° C. nitrogen drying gas temperature, 30 L/min. nitrogen drying gas flow rate, 75 μm internal diameter fused silica restrictor, 5-cm long fused silica restrictor, and 0.45 μm Nylon powder-collection filter.

The resulting dry powder fine particle fraction of less than 5.8 μm was 17%, and resulted in a deposition of 0.28+/−0.03 mg of THC into the lung/pharynx region of the respiratory system, as modeled and fractionated by Andersen cascade impaction (ACI) and assayed by high performance liquid chromatography (HPLC) (see FIG. 3.)

### Example 4

An aliquot (30 mg) of the powder from Example 1 was compressed into a circular wafer using an International Crystal Laboratories E-Z Press equipped with a 7 mm diameter mold by application of 800 psig for 1 minute. An aliquot of limonene (10 μl, 61.8 μmol) was allowed to soak in to the wafer by slow application with a pipet.

The specific embodiments and examples described in the present disclosure are illustrative only in nature and are not limiting of the invention defined by the following claims. Further aspects, embodiments and advantages of the dry powders, wafers and methods of the present invention will be apparent in view of the present disclosure and are encompassed within the following claims.

### REFERENCES

Agarkhedkar, S.; MVDP author group; Cape, S.; Chaudhari, A.; Vaidya V.; Mulay R.; Shermer, C.; Collins, M.; Andersen, R.; Kulkarni, P. S.; Winston, S.; Sievers, R.; Dhere, R. M.; Gunale, B.; Powell, K.; Rota, P. A.; Papania, M. “Safety and immunogenicity of dry powder measles vaccine administered by inhalation: a randomized controlled Phase I clinical trial.” Vaccine. 2014, 32, 6791-6797.
Agurell, S.; Halldin, M.; Lindgren, J. E.; Ohlsson, A.; Widman, M.; Gillespie, H.; Hollister, L. “Pharmacokinetics and metabolism of delta-1-tetrahydrocannabinol and other cannabinoids with emphasis on man.” Pharmacol Rev. 1986, 38, 21.
Andrade-Lima, M.; Pereira, L. F. F.; Fernandes, A. L. G. “Pharmaceutical equivalence of the combination formulation of budesonide and formoterol in a single capsule with a dry powder inhaler.” J. Bras. Pneumol. 2012, 38, 748-756.
Brown, D. T. “The therapeutic potential for cannabis and its derivatives.” In: Brown D T, ed. Cannabis: The Genus Cannabis. Amsterdam: Harwood Academic Publishers, 1998, 175-222.
Chiu, P.; Olsen, D. M.; Borys, H. K.; Karler, R.; Turkanis, S. A. “The influence of cannabidiol and delta-9-tetrahydrocannabinol on cobalt epilepsy in rats.” Epilepsia. 1979, 20, 365-375.
Consroe, P.; Benedito, M. A.; Leite, J. R.; Carlini, E. A.; Mechoulam, R. “Effects of cannabidiol on behavioral seizures caused by convulsant drugs or current in mice.” Eur J Parmacol. 1982, 83, 293-298.
Consroe, P. “Brain cannabinoid systems as targets for the therapy of neurological disorders.” Neurobiol Dis. 1998, 5, 534-551.
Drooge, D. J.; Hinrichs, W. L. J.; Frijlink, H. W. “Incorporation of lipophilic drugs in sugar glasses by lyophilization using a mixture of water and tertiary butyl alcohol as solvent.” J. Pharm. Sci. 2004, 93, 713-725.
El Sohly, M. A.; Little, T. L. Jr.; Hikal, A.; Harland, E.; Stanford, D. F.; Walker, L. “Rectal bioavailability of delta-9-tetrahydrocannabinol from various esters.” Pharm Biochem Behav. 1991, 40, 497-502.
Elzinga, S.; Ortiz, O.; Raber, J. C. “The conversion and transfer of cannabinoids from cannabis smoke stream in cigarettes.” Nat Prod Chem Res. 2015, 3.
Fairbairn, J. W.; Liebmann, J. A.; Rowan, M. G. “The stability of cannabis and its preparations on storage.” J Pharm Pharmac. 1976, 28, 1-7.
Fuentes, J. A.; Ruiz-Gayo, M.; Manzanares, J.; Reche, I.; Corchero, J. “Cannabinoids as potential new analgesics.” Life Sci. 1999, 822, 237-242.
Green, K. “Marijuana smoking vs cannabinoids for glaucoma therapy.” Arch Ophthalmol. 1998, 116, 1433-1437.
Hartel, C. R. “Therapeutic uses of cannabis and cannabinoids.” In: Kalant H, Corrigall W A, Hall W, Smart R G, eds. The Health Effects of Cannabis. Toronto, CAMH. 1999, 461-474.
Healy, A. M.; McDonald, B. F.; Tajber, L.; Corrigan, O. I. “Characterisation of excipient-free nanoporous microparticles (NPMPs) of bendroflumethiazide.” Euro J Pharm Biopharm. 2008, 69, 1182-1186.
Holdcroft, A.; Smith, M.; Jacklin, A.; et al. “Pain relief with oral cannabinoids in familial Mediterranean fever.” Anaesthesia. 1997, 52, 483-486.
House of Lords, Select Committee on Science and Technology, Ninth Report. Cannabis: The Scientific and Medical Evidence. London: Parliament Stationery Office, 1998.
Huestis, M. A. “Human Cannabinoid Pharmacokinetics.” Chem Biodivers. 2007, 4, 1770-1804.
Jain, A. K.; Ryan, J. R.; McMahon, F. G.; Smith, G. “Evaluation of intramuscular levonantrodol and placebo in acute postoperative pain.” J Clin Pharmacol. 1981, 21, 320S-6S.
Jones, R. T.; Benowitz, N. “The 30-day trip—Clinical studies of cannabis tolerance and dependence.” In: Braude M C, Szara S, eds. Pharmacology of Marijuana, vol 2. New York: Academic Press. 1976, 627-642.
Joy, J. E.; Watson, S. J. Jr.; Benson, J. A. Jr.; eds. Marijuana and Medicine: Assessing the Science Base. Washington: National Academy Press. 1999.
Kalant, H. “Medicinal use of cannabis: history and current status.” Pain Res Manage. 2001, 6, 80-91.
Karler, R.; Turkanis, S. A. “The cannabinoids as potential antiepileptics.” J Clin Pharmacol. 1981, 21, 437S-448S.
Kim, K. R.; Yoon, H. R. “Rapid screening for acidic non-steroidal anti-inflammatory drugs in urine by gas chromatography-mass spectrometry in the selected-ion monitoring mode.” J Chromatogr B. 1996, 682, 55.
Luo, S.; Levine, R. L. “Methionine in proteins defends against oxidative stress.” Faseb J. 2009, 23, 464-472.
Mattes, R. D.; Shaw, L. M.; Edling-Owens, J.; Engelman, K.; El Sohly, M. A. “Bypassing the first-pass effect for the therapeutic use of cannabinoids.” Pharm Biochem Behav. 1993, 44, 745-747.
Mattes, R. D.; Engelman, K.; Shaw, L. M.; ElSohly, M. “Cannabinoids and appetite stimulation.” Pharmacol Biochem Behav. 1994, 49, 187-195.
Mendelson, J. H. “Marijuana use: Biologic and behavioral aspects.” Postgrad Med. 1976, 60, 111-115.
Meng, I. D.; Manning, B. H.; Martin, W. J.; Fields, H. L. “An analgesia circuit activated by cannabinoids.” Nature. 1998, 395, 381-383.
Mikuriya, T. H. “Marijuana in medicine: past, present and future.” Calif Med. 1969, 110, 34-40.
Milstein, S. L.; MacCannell, K.; Karr, G.; Clark, S. “Marijuana-produced changes in pain tolerance. Experienced and non-experienced subjects.” Int Pharmacopsychiatry. 1975, 10, 177-182.
Noyes, R. Jr.; Brunk, S. F.; Avery, D. H.; Canter, A. “The analgesic properties of delta-9-tetrahydrocannbinol and codeine.” Clin Pharmacol Ther. 1975, 18, 84-89.
Ohlsson, A.; Lindgren, J. E.; Wahlen, A.; Agurell, S.; Hollister, L. E.; Gillespie, H. K. “Plasma delta-9-tetrahydrocannabinol concentrations and clinical effects after oral and intravenous administration and smoking.” Clin Pharmacol. 1980, 28, 409.
Ohlsson, A.; Lindgren, J. E.; Wahlen, A.; Agurell, S.; Hollister, L. E.; Gillespie, H. K. “Single dose kinetics of deuterium labelled delta-1-tetrahydrocannabinol in heavy and light cannabis users.” Biomed Environ Mass Spectrom. 1982, 9, 6.
Ohlsson, A.; Agurell, S.; Londgren, J. E.; Gillespie, H. K.; Hollister, L. E. “Pharmacokinetics and Pharmacodynamics of Psychoactive Drugs.” Barnett, G, Chiang, C N, eds. Mosby Yearbooks, St. Louis. 1985, p. 75.
Perez-Reyes, M.; Lipton, M. A.; Timmons, M. C.; Wall, M. E.; Brine, D. R.; Davis, K. H. “Pharmacology of orally administered 9-tetrahydrocannabinol.” Clin Pharmacol Ther. 1973, 14, 48.
Plasse, T. F.; Gorter, R. W.; Krasnow, S. H.; Lane, M.; Shepard, K. V.; Wadleigh, R. G. “Recent clinical experience with Dronabinol.” Pharm Biochem Behav. 1991, 40, 695-700.
Raft, D.; Gregg, J.; Ghia, J.; Harris L. “Effects of intravenous tetrahydrocannabinol on experimental and surgical pain. Psychological correlates of the analgesic response.” Clin Pharmacol Ther. 1977, 21, 26-33.
Sellers, S. P.; Clark, G. S.; Sievers, R. E.; Carpenter, J. F. “Dry powders of stable protein formulations from aqueous solutions prepared using supercritical CO₂-assisted aerosolization.” J. Pharm. Sci. 2001, 90, 785-797.
Stinchcomb, A. L.; Valiveti, S.; Hammell, D. C.; Ramsey, D. R. “In vitro/in vivo correlation studies for transdermal delta-8-THC development.” J Pharm Pharmacol. 2004, 56, 291.
Tashkin, D. P. “Cannabis and immunity.” In: Kalant H, Corrigall W A, Hall W, Smart R G, eds. The Health Effects of Cannabis. Toronto: CAMH. 1999, 313-345.
Trofin, I. G.; Dabija, G.; Vaireanu, D.-I.; Filipescu, L. “The influence of long-term storage conditions on the stability of cannabinoids derived from cannabis resin.” Rev Chim. 2012, 63, 422-427.
Walton, R. P. “Marijuana. America's new drug problem.” New York: JB Lippincott, 1938.
Widman, M.; Nilsson, I. M.; Nilsson, J. L.; Agurell, S.; Leander, K. “Metabolism of cannabis, IX, Cannabinol: Structure of a major metabolite formed in rat liver.” Life Sci. 1971, 10, 157-162.
Wolff, R. K.; Niven, R. W. “Generation of aerosolized drugs.” J. Aerosol Med. 1994, 7, 89-106.

1. A dry powder comprising at least one cannabinoid, a polymer binding agent, a dispersing agent, and a bulking agent, and optionally an antioxidant, the dry powder formed by carbon dioxide-assisted nebulization of a solution comprising at least one cannabinoid, a polymer binding agent, a dispersing agent, and a bulking agent, and optionally an antioxidant and drying droplets formed by the nebulization in a flowing stream of gas to produce a dry powder, wherein the dry powder has an aerodynamic particle distribution effective for delivery of the dry powder by respiration into a lung of a patient.

2. (canceled)

3. A dry powder according to claim 1, wherein the solution further comprises a volatile component comprising one or more of ammonium carbonate, ammonium bicarbonate, triethylammonium bicarbonate, trimethylammonium carbonate, trimethylammonium bicarbonate, ammonium acetate, triethylammonium acetate, trimethylammonium acetate, ammonium formate, trimethylammonium formate, and triethylammonium formate.

4. A dry powder according to claim 1, wherein the polymer binding agent comprises one or more of polyvinylpyrrolidone, polyethylene glycol, poly(lactic-co-glycolic) acid, polyvinyl alcohol, polyacrylic acid, N-(2-hydroxypropyl) methacrylamide, polyoxazoline, polyphosphazene, xanthan gum, gum Arabic, pectin, chitosan derivative, dextran, carrageenan, guar gum, cellulose ether, hyaluronic acid, albumin, and starch.

5. A dry powder according to claim 1, wherein the dispersing agent comprises one or more of methionine, alanine, arginine, asparagine, aspartic acid, cysteine, glutamic acid, glutamine, glycine, histidine, isoleucine, leucine, lysine, phenylalanine, proline, serine, threonine, tryptophan, tyrosine, valine, vitamin A, vitamin C, vitamin E, alpha-carotene, astaxanthin, beta-carotene, canthaxanthin, lutein, lycopene, zeaxanthin, flavonoid, cinnamic acid, chicoric acid, chlorogenic acid, rosmarinic acid, curcumin, xanthone, eugenol, citric acid, oxalic acid, and lipoic acid.

6. A dry powder according to claim 1, wherein the bulking agent comprises one or more of mannitol, glucose, galactose, fructose, mannose, allose, altrose, fucose, gulose, sorbose, tagatose, arabinose, lyxose, rhamnose, ribose, xylose, erythrose, threose, lactose, maltose, sucrose, trehalose, lactulose, cellobiose, chitobiose, allolactose, sucralose, mannobiose, maltitol, sorbitol, xylitol, erythritol, isomalt, arabitol, ribitol, galactitol, fucitol, iditol, myo-inositol, volemitol, lactitol, maltotriitol, maltotetraitol, maltodextrin, and polyglycitol.

7. A dry powder according to claim 1, wherein the cannabinoid comprises cannabidiol.

8. A dry powder according to claim 1, wherein the cannabinoid is delta-9-tetrahydrocannabinol.

9. A dry powder according to claim 1, comprising at least two cannabinoids.

10. A dry powder according to claim 3, wherein the polymer binding agent comprises polyvinylpyrrolidone, polyethylene glycol and/or poly(lactic-co-glycolic) acid, the volatile component comprises a volatile salt, the dispersing agent comprises an amino acid, and the bulking agent comprises a non-hygroscopic polyol.

11. A dry powder according to claim 3, wherein the polymer binding agent comprises polyvinylpyrrolidone, the volatile component comprises ammonium bicarbonate or ammonium carbonate, the dispersing agent comprises methionine, and the bulking agent comprises mannitol.

12. A dry powder according to claim 1, comprising at least 30% of particles having a size of less than 5.8 μm as modeled by an Andersen Cascade Impactor according to US Pharmacopeia <601>.

13. A wafer formed by compressing a dry powder according to claim 1.

14. A wafer according to a claim 13, comprising a terpene.

15. A wafer according to claim 14, wherein the terpene is infused in the wafer.

16. A method of preparing the dry powder of claim 1, the method comprising subjecting a solution of a volatile component, a cannabinoid, a polymer binding agent, a dispersing agent, and a bulking agent, and optionally an antioxidant, to carbon dioxide-assisted nebulization, and drying droplets formed by the nebulization in a flowing dry gas stream to form a dry powder, wherein the dry powder has an aerodynamic particle distribution effective for delivery of the dry powder by respiration into a lung of a patient.

17. A method according to claim 16, wherein the flowing gas stream comprises gaseous nitrogen or gaseous carbon dioxide.

18. A method according to claim 16, wherein the dry powder comprises at least 30% of particles having a size of less than 5.8 μm as modeled by an Andersen Cascade Impactor according to US Pharmacopeia <601>.

19. A method of increasing the bioavailability and/or storage stability of a cannabinoid, the method comprising subjecting a solution of a volatile component, a cannabinoid, a polymer binding agent, a dispersing agent, and a bulking agent, and optionally an antioxidant, to carbon dioxide-assisted nebulization, and drying droplets formed by the nebulization in a flowing dry gas stream to form a dry powder according to claim 1.

20. A method according to claim 19, wherein the dry powder has an aerodynamic particle distribution effective for delivery of the dry powder by respiration into a lung of a patient.

21. A dry powder according to claim 1, wherein the flowing stream of gas has a temperature of above ambient to about 40° C.

22. A dry powder according to claim 1, comprising from about 10 to about 40 wt % cannabinoid, from about 5 to about 20 wt % polymer binding agent, a dispersing agent, and from about 20 to about 40 wt % bulking agent.
